(19) United States
(12) Patent Application Publication
(10) Pub. No.: US 2015/0204770 A1
(43) Pub. Date: Jul. 23, 2015

(54) Pore Analysis

(71) Applicant: University of Plymouth, Plymouth, Devon (GB)

(72) Inventors: Christopher Mark Gribble, Plymouth (GB); Giuliano Maurizio Laudone, Plymouth (GB); Philip Martin Gerstner, Plymouth (GB); Graham Peter Matthews, Plymouth (GB); Joseph William Badenoch Matthews, Plymouth (GB)

(73) Assignee: UNIVERSITY OF PLYMOUTH, Plymouth, Devon (GB)

(21) Appl. No.: 14/413,167

(22) PCT Filed: Jul. 2, 2013
(86) PCT No.: PCT/GB2013/051753

§ 371 Date: Jan. 6, 2015

(30) Foreign Application Priority Data
Jul. 6, 2012 (GB) 1212078.8

(51) Int. Cl. G01N 15/08; G06F 17/50; G06F 17/10; G01N 33/34

(52) U.S. Cl. CPC G01N 15/088; G01N 33/34; G06F 17/50; G06F 17/10

(57) ABSTRACT

A method of producing a product having a predetermined porosity comprises the following steps: conducting a porosity test upon a test sample; producing a 3 dimensional model of voids interconnected by throats; deriving an intrusion curve for the model; comparing the derived, intrusion curve for the model with an intrusion curve showing the output of the porosity test; iteratively modifying the model until the derived intrusion curve for the model matches that for the output of the porosity test; making adjustments to at least one parameter of the model to adjust the porosity characteristics of the model to the predetermined porosity characteristics; and modifying the test sample in accordance with the adjustments.

This invention relates to pore analysis, and in particular to techniques whereby the structure of a porous material can be modelled, and whereby a product having a desired, predetermined porosity or porosity characteristics can be designed.

Porous materials surround us and are present, or are in widespread use, in a number of technologies upon which we rely. By way of example, the oil and gas located within subsurface reservoirs is usually located within porous rocks. In order to maximise the efficiency with which oil and gas can be extracted, information about the porosity of the rock formations within which the oil and gas is located is important. In printing technologies, the porosity of coatings provided upon paper, card or other media to which printing ink or the like is delivered is important as it impacts significantly upon the quantity of ink that must be applied to achieve a desired appearance, and it impacts upon the manner in which the ink dries or cures, and so is of importance in determining subsequent processing steps. These are merely two examples of technologies in which knowledge of the porosity of a material is of importance. There are numerous other applications, and the invention is not restricted in this regard.

Studying the structure of a porous material is very difficult. Whilst external surfaces of a material can be studied reasonably easily, for example by visual inspection using a microscope or other magnifying means in order to allow a detailed study thereof, and this can allow the structure of the material at the surface to be ascertained, observing the presence of voids and the degree to which those voids are interconnected at the surface, extrapolating these findings to provide an indication of the internal structure of the material, and the manner by which a fluid will flow through the material, is difficult.

One technique by which the porosity characteristics of a material can be measured, or information about the pore structure can be derived, involves forcing a fluid into the material. By measuring the amount of intrusion of the fluid into the material at different applied fluid pressures, data representative of the pore structure can be ascertained. The applied fluid pressures are typically very high. By way of example, the applied fluid pressure may reach 400 MPa. When conducting this type of test, the fluid used should be a non-wetting fluid with respect to the solid phase and fluid, for example, already present within the pores. By way of example, a test sample material may be evacuated and, subsequently, mercury used as the fluid that is forced into the pores of the material.

Whilst mercury porosimetry techniques are well established, interpretation of the data achieved by the use of such techniques is very difficult. Typically, a graph of intrusion of the fluid against applied differential pressure is plotted. Generally, this will provide an S-shaped curve. The steepest part of the curve is generally assumed to provide an indication of or guide to the porosity of the material. However, the information so derived is difficult to interpret accurately, and so is of limited applicability.

A methodology for interpreting the output of porosimetry techniques is described in, for example, Matthews, G. P., Ridgway, C. J. and Spearing, M. C. (1995) Void space modeling of mercury intrusion hysteresis in sandstone, paper coating, and other porous media. Journal of Colloid and Interface Science, 171, 8-27, Bodurtha, Pa., Matthews, G. P., Kettle, J. P. and Roy, I. M. (2005) Influence of anisotropy on the dynamic wetting and permeation of paper coatings. J. Colloid and Interface Science, 283, 171-189, and Gribble, C. M., Matthews, G. P., Laudone, G. M., Turner, A., Ridgway, C. J., Schoelkopf, J. and Gane, P. A. C., (2011) Porometry, porosimetry, image analysis and void network modelling in the study of the pore-level properties of filters. Chem. Eng. Sci., 66(16): 3701-3709. This methodology involves generating a 3-dimensional model in the form of a network of voids interconnected with one another by throats, deriving an intrusion curve for a notional material having that structure, comparing the derived intrusion curve for the model with that for the output data from a test sample, modifying the model, for example by changing the sizes and/or numbers of the voids and throats, and repeating the derivation step until the derived intrusion curve for the model closely matches that for the test data. After completion of this process, if a porosimetry test were conducted upon a test sample manufactured in accordance with the model design, the test output and associated intrusion curve would match those produced from the original test.

For simplicity, in the model, the voids are conveniently of cubic shape, and the throats are of cylindrical form. However, modification of the model to use other shapes may be possible.

The model produced using this technique is advantageous in that it allows a user to visualise a structure having the porosity attributes of the test sample.

The methodology outlined above and described in greater detail in the papers referenced above is a considerable step forwards compared to the prior techniques of outputting the test results by plotting the intrusion and pressure differential on a graph and using the graph to determine a porosity value for the test sample. However, it is of limited value where there is a need to design a product having predetermined porosity characteristics.

It is an object of the invention, therefore, to provide a method whereby a sample product having predetermined porosity characteristics can be designed and manufactured.

According to a first aspect of the invention, therefore, there is provided a method of producing a product having a predetermined porosity, the method comprising the steps of:
conducting a porosity test upon a test sample;
producing a 3 dimensional model of voids interconnected by throats;
deriving an intrusion curve for the model;
comparing the derived intrusion curve for the model with an intrusion curve showing the output of the porosity test;
iteratively modifying the model until the derived intrusion curve for the model matches that for the output of the porosity test;
making adjustments to at least one parameter of the model to adjust the porosity of the model to the predetermined porosity; and
modifying the test sample in accordance with the adjustments.

The at least one parameter of the model preferably comprises at least one of the edge length or size of voids, the throat diameter of throats, the position and spacing of voids and throats, and the number of throats attached to each void. The parameters may be adjusted in combination, if desired.

The at least one parameter may be adjusted throughout the model. Alternatively, it may be adjusted in, for example, just selected regions of the model.

If desired, the step of adjusting may be repeated until such time as the predetermined porosity has been achieved. The adjusting step may be automated, if desired, in the sense that a user may input a value for the predetermined porosity, and the adjusting step may comprise iteratively adjusting the at least one parameter until the predetermined porosity is achieved.

The step of modifying the test sample may comprise, for example, mechanically and/or chemically modifying the pore structure of the test sample to modify, for example, its packing density, particle size distribution, or by changing colloidal interactions or making intra particle modifications.

One porosity parameter of use in certain industries relates to the connectivity between pores. For example, knowledge about the number or concentration of pores within a sample which have just one connection thereto (so called ‘ink bottle pores’) is of importance in the cement industry as such pores are able to store water. During cold weather, freeze-thaw conditions resulting in expansion and contraction of the water stored or contained within such pores can cause premature weathering of materials containing cement. By acquiring knowledge regarding the number or concentration of such pores, the risk of such premature weathering can be avoided or reduced through ensuring that the materials used are suitable for use in the environmental conditions to which the material is likely to be exposed.

According to another aspect of the invention, therefore, there is provided a method of modelling the presence of ink bottle pores in a test sample, comprising the steps of:
conducting a porosity test upon a test sample;
producing a 3 dimensional model of voids interconnected by throats;
deriving an intrusion curve for the model;
comparing the derived intrusion curve for the model with an intrusion curve showing the output of the porosity test;
iteratively modifying the model until the derived intrusion curve for the model matches that for the output of the porosity test; and
outputting information regarding the number, location and/or concentration of voids having a number of throats connected thereto lower than a predetermined number.

Conveniently, the aforementioned predetermined number of throats is set so that information regarding the number, location and/or concentration of voids having just one throat connected thereto is output. In this way, information regarding ink bottle pores can be ascertained.

The method according to the second aspect of the invention may be used in conjunction with that according to the first aspect of the invention to design and produce a sample with, for example, fewer ink bottle pores.

There is a desire to be able to model the impact of the fluid content of a porous material upon the thermal conductivity of the material, as the presence or absence of fluid from some of the pores thereof will impact upon the thermal conductivity characteristics of the sample.

According to another aspect of the invention, therefore, there is provided a method of modelling the impact of the porosity of a material on the thermal conductivity thereof comprising the steps of:
conducting a porosity test upon a test sample; producing a 3 dimensional model of voids interconnected by throats;
deriving an intrusion curve for the model;
comparing the derived intrusion curve for the model with an intrusion curve showing the output of the porosity test;
iteratively modifying the model until the derived intrusion curve for the model matches that for the output of the porosity test;
simulating the location of a wetting fluid within the voids and/or throats of the model; and
outputting information regarding the impact of the presence or absence of wetting fluid from the voids and/or throats upon the thermal conductivity of the sample.

The invention will further be described, by way of example, with reference to the accompanying drawings, in which:

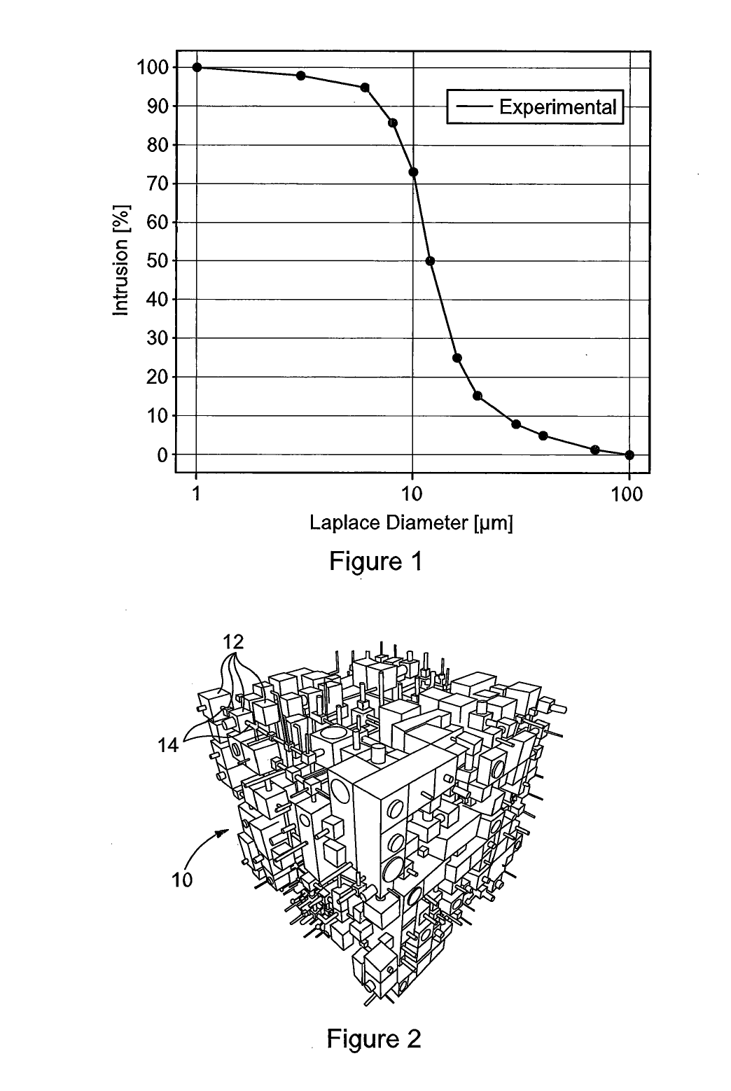

FIG. 1 is a diagram representing an intrusion curve for a test sample; and

FIG. 2 is a diagram illustrating a 3 dimensional model of voids interconnected by throats.

Referring firstly to FIGS. 1 and 2, a method for producing a product having desired porosity characteristics is illustrated, the method involving conducting tests upon a sample material, for example by using a mercury porosimetry process to produce data representative of the intrusion of a fluid such as mercury into the sample material at different applied pressures. FIG. 1 illustrates, diagrammatically, the output from such tests illustrated in the form of an intrusion curve. It will be appreciated that the form of the intrusion curve is characteristic of the material upon which the test has been performed.

As well as conducting tests on a sample material to ascertain porosity information therefrom, a 3 dimensional model 10 indicative of the structure of the sample material is produced. This is achieved by constructing a 3 dimensional model consisting of a series of voids 12 (representative of the pores of the sample material) interconnected by a series of throats 14 (representing the interconnections between the pores). For convenience and simplicity, the voids 12 are each of cubic form and the throats 14 are of cylindrical form. It will be appreciated, however, that more complex shapes may be used if desired.

Once the model 10 has been constructed, calculations are undertaken to ascertain data indicative of the porosity characteristics of the model. This data can be output in the form of an intrusion curve. It will be appreciated that by comparing the intrusion curve of the model with that derived from the tests conducted upon the sample material, it can be determined whether or not the model 10 is representative of the sample material. The model 10 does not necessarily provide an accurate reconstruction of the structure of the sample material, but rather provides a representation of a sample material having the same porosity characteristics as the sample material. Most likely, the porosity of the initial form of the model 10 will differ significantly from that of the sample material, and so the model 10 will require some adjustment in order to properly represent the sample material. The adjustment may involve modifying parameters of the model, for example modifying the sizes of the voids 12 (for example by specifying a different value for the length dimensions thereof) or the throats 14 (for example by specifying a different value for the diameter thereof).

By repeatedly modifying the parameters of the model 10 and repeating the calculations to ascertain the porosity characteristics of the model 10, it will be appreciated that the model 10 can be modified until such time as the intrusion curve for the model 10 matches that for the sample material, at which point the model 10 will provide an accurate representation of the porosity characteristics of the sample material.

As described hereinbefore, such a technique is described in, for example, Matthews, G. P., Ridgway, C. J. and Spearing, M. C. (1995) Void space modeling of mercury intrusion hysteresis in sandstone, paper coating, and other porous media. Journal of Colloid and Interface Science, 171, 8-27, Bodurtha, Pa., Matthews, G. P., Kettle, J.P. and Roy, I. M. (2005) Influence of anisotropy on the dynamic wetting and permeation of paper coatings. J. Colloid and Interface Science, 283, 171-189, and Gribble, C. M., Matthews, G. P., Laudone, G. M., Turner, A., Ridgway, C. J., Schoelkopf, J. and Gane, P. A. C., (2011) Porometry, porosimetry, image analysis and void network modelling in the study of the pore-level properties of filters. Chem. Eng. Sci., 66(16): 3701-3709.

In accordance with one aspect of the invention, the method outlined hereinbefore may be used in the design and production of a product having predetermined porosity characteristics. This is achieved by, after production of the model 10 which accurately represents the porosity characteristics of the sample material, adjusting the model 10 to modify the porosity characteristics thereof until desired porosity characteristics have been achieved. This may be achieved by adjusting the same parameters as were adjusted during the creation of the model 10. Thus, for example, the parameters which are adjusted may include at least one of the edge length or size of voids 12, the throat diameter of throats 14, the position and spacing of voids 12 and throats 14, and the connectivity between the voids 12, in other words the number of throats 14 attached to each void 12. These parameters may be adjusted in combination, if desired. The parameters may be adjusted throughout the model or, alternatively, they may be adjusted in, for example, just selected regions of the model.

By repeatedly adjusting the values of the parameters, and monitoring the effect of the adjustments upon the calculated porosity, the parameter values may be adjusted until such time as the predetermined porosity characteristics have been achieved.

It is envisaged that the adjusting step will be a manual operation, with a user deciding which parameter values to adjust, and the degree by which they are adjusted, the user determining whether the effect of the adjustment is sufficient to result in the desired, predetermined porosity characteristics have been attained. However, the adjusting step may, if desired, be automated in the sense that a user may input a value for the predetermined porosity, and the adjusting step may comprise automatically, iteratively adjusting the at least one parameter until the predetermined porosity characteristics are achieved.

Once the model has been adjusted in this manner, data is output providing an indication to the user of how the sample material should be modified in order to result in the porosity characteristics of the sample material matching the predetermined porosity characteristics. The techniques by which the sample material may be modified include mechanically and/or chemically modifying the pore structure of the sample material to modify, for example, its packing density, particle size distribution, or by changing colloidal interactions or making intra particle modifications. These techniques are well known and so the matter in which they may be embodied is not described herein in further detail.

Thus, for example, if the void dimensions of the model 10 were increased in order to result in the desired porosity characteristics, the production step may involve chemically treating the sample material to increase the pore sizes thereof.

It will be appreciated that certain parameters may be interrelated. For example, if the void sizes are increased, the separation between adjacent voids will have to be reduced if the concentration of voids is to be held constant.

It will be appreciated that in accordance with this aspect of the invention, a product may be produced having desired porosity parameters or characteristics, something that has not been easy to achieve hitherto.

As mentioned hereinbefore, one porosity characteristic which is of importance in certain technologies relates to the presence of so-called ink bottle pores in a material. This terminology is used to refer to pores having just one connection thereto. Whilst reference is made herein to ink-bottle pores, it will be appreciated that pores with limited numbers of connections, for example pores with just two connections thereto, may have similar effects to those with a single connection. There is a desire to be able to ascertain the presence of such pores, and their concentration within a sample material.

In accordance with another aspect of the invention, therefore, after having produced the model 10 which is representative of the sample material as outlined hereinbefore, the model 10 is studied to ascertain how many voids 12 are present to which only a single throat 14, or fewer than a predetermined number of throats 14, is connected. Not only is the model 10 able to provide an indication of the number and/or concentration of such voids 12, but also their location within the model 10 may be highlighted to that a user can inspect the modelled structure and come to a view over the likely impact of the presence of such pores in the sample material.

The model 10 may further permit the total volume of the voids 12 with only one throat 14, or fewer than a predetermined number of throats 14, to be calculated.

As mentioned hereinbefore, information about the number and/or concentration of ink-bottle pores, and about the total volumes of such pores, is of use in the cement industry. It may also be of use in other technologies.

The model 10 may further be used to provide an indication of the effect of the presence of fluid upon the thermal conductivity of the sample material. Once the model 10 has been produced and modified so that it provides an accurate representation of the characteristics of the sample material, calculations can be undertaken to provide an indication of the volume of the sample material which will contain a wetting fluid when that fluid is applied at a given pressure. The model may also provide a representation of the types of pores and throats containing the fluid. With knowledge of the thermal conductivity of the fluid and of the sample material, calculations can be undertaken in accordance with the invention to ascertain the effect of the presence of the fluid upon the thermal conductivity of the sample material containing the fluid.

Whilst specific aspects and applications of the invention have been described hereinbefore, it will be appreciated that a wide range of modifications and alterations may be made without departing from the scope of the invention.

1. A method of producing a product having a predetermined porosity, the method comprising the steps of
conducting a porosity test upon a test sample
producing a 3 dimensional model of voids interconnected by throats;
deriving an intrusion curve for the model,
comparing the derived intrusion curve for the model with an intrusion curve showing the output of the porosity test;
iteratively modifying the model until the derived intrusion curve for the model matches that for the output of the porosity test;
making adjustments to at least one parameter of the model to adjust the porosity characteristics of the model to the predetermined porosity characteristics; and
modifying the test sample in accordance with the adjustments.

2. The method according to claim 1, wherein the at least one parameter of the model comprises at least one of the edge length or size of voids, the throat diameter of throats, the position and spacing of voids and throats, and the number of throats attached to each void.

3. The method of claim 2, wherein two or more of the parameters are adjusted in combination.

4. The method according to claim 1, wherein the at least one parameter is adjusted throughout the model.

5. The method according to claim 1, wherein the at least one parameter is adjusted in just selected regions of the model.

6. The method according to claim 1, wherein the step of adjusting is repeated until such time as the predetermined porosity characteristics have been achieved.

7. The method according to claim 6, wherein the adjusting step is automated, a user inputting a value for the predetermined porosity, and the adjusting step comprising iteratively adjusting the at least one parameter until the predetermined porosity characteristics are achieved

8. The method of claim 1, wherein the step of modifying the test sample comprises mechanically and/or chemically modifying the pore structure of the test sample to modify its packing density and/or particle size distribution, and/or by changing colloidal interactions and/or making intra particle modifications.

9. A method of modelling the presence of ink bottle pores in a test sample, comprising the steps of:
conducting a porosity test upon a test sample;
producing a 3 dimensional model of voids interconnected by throats;
deriving an intrusion curve for the model;
comparing the derived intrusion curve for the model with an intrusion curve showing the output of the porosity test;
iteratively modifying the model until the derived intrusion curve for the model matches that for the output of the porosity test; and
outputting information regarding the number, location and/or concentration of voids having a number of throats connected thereto lower than a predetermined number.

10. The method according to claim 9, wherein the aforementioned predetermined number of throats is set so that information regarding the number, location and/or concentration of voids having just one throat connected thereto is output.

11. The method of claim 9, further comprising the steps of:
making adjustments to at least one parameter of the model to adjust the number, location and/or concentration of voids having a number of throats connected thereto lower than a predetermined number of the model to a predetermined number; and
modifying the test sample in accordance with the adjustments.

12. A method of modelling the impact the porosity of a material on the thermal conductivity thereof comprising the steps of:
conducting a porosity test upon a test sample;
producing a 3 dimensional model of voids interconnected by throats;
deriving an intrusion curve for the model;
comparing the derived intrusion curve for the model with an intrusion curve showing the output of the porosity test;
iteratively modifying the model until the derived intrusion curve for the model matches that for the output of the porosity test;
simulating the location of a wetting fluid within the voids and/or throats of the model; and
outputting information regarding the impact of the presence or absence wetting fluid from the voids and/or throats upon the thermal conductivity of the sample.
